(19) United States
(12) Patent Application Publication
(10) Pub. No.: US 2005/0158393 A1
(43) Pub. Date: Jul. 21, 2005

(54) Colloidal metal labeled microparticles and methods for producing and using the same

(76) Inventors: Phillippe Reb, Chaumes en Brie (FR); Laurent Domas, Paris (FR); Egisto Boschetti, Croissy sur Seine (FR); Marie-Paule Leroy-Landercy, Paris (FR)

Correspondence Address: JONES DAY, 51 Louisiana Aveue, N.W, WASHINGTON, DC, 20001-2113, US

(21) Appl. No.: 11/030,182

(22) Filed: Jan. 7, 2005

Related U.S. Application Data
(63) Continuation of application No. 09/945,793, filed on Sep. 5, 2001, now abandoned.
Application No. 09/945,793 is a continuation of application No. PCT/IB01/01266, filed on Jun. 8, 2001.

(51) Int. Cl. A61K 33/24; A61K 9/14

(52) U.S. Cl. 424/489; 424/617

(57) ABSTRACT

The present invention relates to polymeric materials that are labeled with colloidal metals, preferably colloidal gold, to processes for producing the labeled polymeric material, and to methods of using the materials in prophylactic, therapeutic and cosmetic applications. Specifically, the invention relates to porous injectable and implantable microparticles, preferably microspheres, that are associated with colloidal metals such that the microparticles are visible or detectable under regular light, by radiological and/or magnetic resonance imaging techniques, or both. The microparticles having colloidal metals are particularly useful for embolization, dermal augmentation and tissue bulking, drug delivery, gene therapy, and other prophylactic, therapeutic or cosmetic medical applications.

### CROSS-REFERENCE TO RELATED APPLICATIONS

This application is a continuation of application Ser. No. 09/945,793, filed Sep. 5, 2001, which, in turn, is a continuation of PCT application No. PCT/IB01/01266, filed Jun. 8, 2001, with the International Bureau as the receiving Office, via the French Intellectual Property Office.

### FIELD OF THE INVENTION

The present invention relates to polymeric materials that are labeled with colloidal metals, to processes for producing the labeled polymeric material, and to methods of using the materials in prophylactic, therapeutic and cosmetic applications. Specifically, the invention relates to porous injectable and implantable microparticles that are associated with colloidal metals, preferably colloidal gold, such that the microparticles are visible or detectable under regular light through naked eye, by radiological imaging techniques, or both. The microparticles having colloidal metals are particularly useful for embolization, dermal augmentation and tissue bulking, drug delivery, gene therapy, and other prophylactic, therapeutic or cosmetic medical applications.

### BACKGROUND OF THE INVENTION

Radiopaque Labeling of Polymeric Implants

The “labeling of” biocompatible polymeric materials, from traditional prosthetic devices to tissue bulking materials to emboli for vascular occlusion, has been a subject of interest since the devices or materials themselves were first introduced. Labeling is useful, if not necessary, to properly detect, control, and/or study the effect of the implanted or injected material. Chemical dyes, magnetic resonance agents, and contrasting/radiopaque agents have all been used to serve such purposes. Radiopaque labeling of polymeric materials, which constitute the vast majority of implanted materials, has received the most attention.

Radiological detectability polymers used as medical implants is limited by the density of the polymers, which is similar to that of the soft tissue because they contain the same elements such as carbon, hydrogen, oxygen and nitrogen. To improve the radio-visibility if the polymers, heavy elements have been incorporated into the polymers to increase the average electron density and specific gravity. The most commonly used heavy elements include iodine, barium, bismuth, zircon, and tantalum. See, e.g., Mottu et al., Investigative Radiology, 34: 323 (1998) (“Mottu”). Some studies have focused on the incorporation of heavy metal salts into the polymers as physical mixtures. A major disadvantage of this method is the non-homogeneity of the mixtures, due to the basic incompatibility between ionic salts and resins. See, e.g., Mottu at 323. Attempts to overcome this drawback include: (a) developing a single phase solution of polymer-radiopaque salt complex via chelation between the heavy metal salts and the polymer backbone and (b) introducing the heavy metals into the initial polymerization suspension such that the metals are bound electrovalently or covalently to the resultant polymer backbone.

Colloidal Metals As Markers in Immunocytochemistry

Colloidal metals, especially colloidal gold, have a long history as staining agents in various applications. In 1857, Faraday speculated that the red color of colloidal gold resulted from the reflection of the light, a property which became the basis for its initial use in light microscopy. Faraday, Philos. Trans. R. Soc. Lond. B. Biol. Sci., 147: 145 (1857). Thiessen proved the particulate nature of colloidal gold in 1942. Thiessen et al., Kolloid Z, 101: 241 (1942) Perhaps the first true applications in cell biology were by Harford et al., J. Biophys. Biochem. Cytol., 3: 749 (1957) and Feldherr et al., J. Biophys. Biochem. Cytol., 12: 640 (1962), who used stabilized colloidal gold as an electron-dense tracer in cellular uptake and micro-injection experiments, respectively.

Since the publication by Faulk and Taylor in 1971 of “An Immunocolloid Method For The Electron Microscope,” Faulk et al., Immunochemistry, 8: 1081 (1971), colloidal metals, especially colloidal gold, have become a very widely used marker in light and electronic microscopy. For example, colloidal gold has been used to detect a wide variety of cellular and extracellular constituents by in situ hybridization, immunogold, lectin-gold, and enzyme-gold labeling. Besides its use in light microscopic immunogold and lectin-gold silver staining, colloidal gold remains the label of choice for transmission electron microscopy studying thin sections, freeze-etch, and surface replicas, as well as for scanning electron microscopy. However, the use of colloidal metal, especially colloidal gold, in vivo, has not been reported. Furthermore, using colloidal metals to label or staining a synthetic polymeric material has not been reported either.

Labeling of Embolization Materials

Therapeutic vascular embolization procedures are used to treat or prevent certain pathological situations in vivo. Most generally they are made using catheters or syringes under imaging control to position solid or liquid embolic agents in the target vessel.

Embolization can be used to occlude vessels of a variety of organs including brain, liver, and spinal cord, which results in reduced blood flow or completely occlusion of the vessels. One application of embolization is to stop or reduce blood flow in hemorrhagic situations. Another application is to stop delivery of vital blood supply and nutrients to tissue, for instance, to reduce or deny blood supply to a solid tumor. In the case of vascular malformations, embolization enables the blood flow to the normal tissue, aids in surgery and limits the risks of hemorrhage. Depending on the pathological conditions, embolization can be used for temporary as well as permanent objectives.

Embolization has been performed with a variety of materials such as small pieces of durable matters, including polyvinyl-alcohol irregular particles, liquid embolic products and more recently with spherical shapes solid hydrogels. All known commercially available embolic material is difficult to follow because they either cannot be seen clearly with the normal light before and during administration or are difficult to be detected after administration. They are relatively transparent most of the time and, due to the small amount used for the procedure the practitioner has some hard time to see the particles during the intervention procedures. Several scientific publications describe methods and products that are bulked in such a way to see them under X-ray, however none described a method to obtain colored beads that can be really seen by the surgeon or radiologist.

U.S. Pat. Nos. 5,635,215 and 5,648,100 disclose an injectable microspheres comprising a hydrophilic acrylic copolymer coated with a cell adhesion promoter and a marking agent. Marking agents described in these patents include chemical dyes, magnetic resonance imaging agents, and contrast agents such as barium or iodine salts. Organic dyes are complex molecules composed of aromatic structures and strong ionic charges. They are known especially in affinity chromatography as ligands for several biological structures. Their major limitation as markers for embolic agents are the possible dye release as a result of the hydrolysis of the dye-embolic material link with subsequent delivery in the blood stream. Another limitation of chemical dyes is that they may be absorbed to certain biological structures or tissue, which may produce undesirable results.

For example, it is well known in affinity chromatography that human albumin interacts strongly in physiological conditions with a dye named Cibacron Blue F3GA.

Thanoo et al. reported, in 1991, the preparation and properties of barium sulphate and methyl iothalamate loaded poly(vinyl alcohol) (PVA) microspheres as radiopaque particulate emboli. Thanoo, et al., Journal of Applied Biomaterials, 2: 67 (1991). The barium sulphate and methyl iothalamate impregnated PVA microspheres reported therein were prepared by the glutaraldehyde cross-linking of an aqueous dispersion of PVA containing the radiopaques in paraffin oil using dioctyl sulfosuccinate as the stabilizing agent and thionyl chloride as the catalyst.

Horák et al., in 1998, reported radiopaque poly(2-hydroxyethyl methacrylate) (HEMA) particles containing silver iodide complexes, which were tested on cell culture. Horák et al., Biomaterials, 19: 1303 (1998). The incorporation of silver iodide complexes inside the poly(HEMA) particles was achieved by first swelling the particles in potassium iodide solution and precipitating the silver iodide complexes using a 30 wt % solution of silver nitrate.

Although the methods mentioned above are efficient for staining of soft embolic spherical agents, such as Embosphere® (a trade name of Biosphere Medical Inc.) or PVA microspheres, they may change the physical properties, such as density and compressibility, of the microspheres. Further, they may not provide good visibility, under regular light by naked eyes, for the particles before and during administration. The use of a coloring agent, such as chemical dye, is another possibility to stain the microspheres. But the risk of this method is the release of dye molecules from the microspheres in vivo, as discussed above.

Therefore, there is a need for a way of labeling implantable or injectable polymeric materials in general, and small tissue bulking or embolic materials in particular, such that the materials can be detected readily under regular light by naked eye that can optionally also be detectable by radiologic imaging techniques. At the same time the labeling should be biocompatible and stable at the implantation or injection site.

### SUMMARY OF THE INVENTION

The present invention provides polymeric materials that are associated with colloidal metal particles, processes for producing the labeled polymeric materials, injectable solutions and kits comprising the materials, and methods of using the materials in prophylactic, therapeutic and cosmetic applications. In one embodiment, the invention encompasses colloidal metals, preferably colloidal gold, containing polymeric materials, preferably porous and/or particular polymeric materials, having the essential functions and properties of the original polymeric materials. The colloidal metal labeled polymeric materials of the present invention are readily detectable or visible under regular light through naked eye. The materials may also optionally be detectable by radio imaging techniques.

In one aspect, the present invention is directed to a polymeric material associated with colloidal metal particles. Preferably, the polymeric material is porous and comprises at least part of the metal particles within the pores therein. The materials are capable of being detected under regular light and/or by naked eye. Optionally, the material may also be detectable by radiological imaging techniques. The materials are further preferably implantable or injectable in humans or animals and are biocompatible and stable, with very little or no release of the metal particles within the body. Such metal containing polymeric materials can either form part of a traditional prosthetic device or part of microparticles that are implantable or injectable for dermal augmentation, tissue bulking or embolization purposes. Because of the metal content, the materials are capable of being detected both under regular light and by radiological imaging techniques, enable better control and manipulation of the material in medical applications.

In a preferred embodiment, the polymeric material is porous and comprises at least part of the colloidal metal particles within the pores therein. The material is preferably selected from the group consisting of acrylics, vinyls, acetals, allyls, cellulosics, polyamides, polycarbonate, polyesters, polyimide, polyolefins, polyurethanes, silicones, styrenics, and polysaccharides. In another preferred embodiment, the polymeric material is implantable into a human.

The present invention also provides a microparticle which comprises a polymeric material associated with colloidal metal particles, wherein the microparticle is suitable for injection or implantation into a human.

In a preferred embodiment of the present invention, the microparticle comprises polymeric material selected from one or more of the group consisting of acrylics, vinyls, acetals, allyls, cellulosics, polyamides, polycarbonate, polyesters, polyimide, polyolefins, polyurethanes, silicones, styrenics, and polysaccharides. In another preferred embodiment, the polymeric material is porous. Further, the porous polymeric material may comprises at least part of the colloidal metal particles within the pores therein.

According to the present invention, the microparticle preferably comprises polymeric material that is an elastomer, a hydrogel, a water swellable polymer, or combinations thereof. More preferably, the polymeric material is an acrylic polymer, such as a trisacryl based acrylic polymer. In a most preferred embodiment, the material comprises a hydrophilic acrylic copolymer that contains, in copolymerized form, about 25 to about 98%, by weight, of a neutral hydrophilic acrylic monomer, about 2 to about 50%, by weight, of a difunctional monomer, and about 0 to about 50%, by weight, of one or more monomers having a cationic charge.

Further, the neutral hydrophilic acrylic monomer is preferably selected from the group consisting of acrylamides, methacrylamides and hydroxymethylmethacrylate; the difunctional monomer is preferably selected from the group consisting of N,N′-methylene-bis-acrylamide, N′,N′-diallyltartradiamide, and glyoxal-bis-acrylamide; and the monomer having a cationic charge is preferably a monomer having a tertiary and/or quaternary amine function.

The microparticle of the present invention may further preferably comprises one or more cell adhesion promoters selected from the group consisting of collagen, gelatin, glucosaminoglycans, fibronectin, lectins, polycations, natural biological cell adhesion agents or synthetic biological cell adhesion agents.

The polymeric material, especially the microparticle, of the present invention may optionally comprise traditional marking agents, such as a chemical dye, a magnetic resonance imaging agent, and/or a contrasting agent.

In yet another preferred embodiment of the present invention, the polymeric material is a poly(vinyl alcohol) (“PVA”), preferably a cross-linked PVA. The polymeric material of the present invention may also be a polymethacrylate, such as poly(methyl methacrylate) or poly(2-hydroxyethyl methacrylate).

In another embodiment of the present invention, the polymeric material is in microparticle form with dimensions ranging from about 1 μm to about 2000 μm. In a preferred embodiment, the microparticles are substantially spherical microspheres with diameters ranging from about 10 μm to about 2000 μm, more preferably, from about 40 μm to about 1200 μm. The microparticle of the present invention is preferably suitable for tissue bulking, dermal augmentation, and/or therapeutic vascular embolization purposes.

The polymeric material of the present invention may contain pores both on the surface and within the body. Preferably, the pores have sizes, measured by the dimensions of the cross sections, ranging from about 1 nm to about 10 μm, more preferably, from about 1 nm to about 1000 nm.

The colloidal metal particles contained within the polymeric material have dimensions ranging from about 1 nm to about 1000 nm and, preferably, from about 1 nm to about 500 nm. The metal is preferably selected from the group consisting of gold, silver, platinum, copper, titanium and chromium. Most preferably, the metal is gold.

In another preferred embodiment, the present invention provides a substantially spherical microparticle, or a microsphere, which comprises a hydrogel associated with colloidal gold particles, wherein the microsphere is suitable for injection or implantation into a human. In a more preferred embodiment, the present invention provides a microsphere having a diameter ranging between about 10 μm and about 2000 μm, useful for embolization, which comprises a hydrophilic acrylic copolymer associated with colloidal gold particles, wherein the hydrophilic acrylic copolymer comprises, in copolymerized form, about 25 to about 98%, by weight, of a neutral hydrophilic acrylic monomer, about 2 to about 50%, by weight, of a difunctional monomer, and about 0 to about 50%, by weight, of one or more monomers having a cationic charge.

The microsphere of the present invention may also comprise one or more cell adhesion promoters selected from the group consisting of collagen, gelatin, glucosaminoglycans, fibronectin, lectins, polycations, natural biological cell adhesion agents or synthetic biological cell adhesion agents. Further, the microsphere may optionally comprise a marking agent selected from the group consisting of dyes, imaging agents, and contrasting agents.

In another aspect, the present invention relates to a process of associating colloidal metal particles with a polymeric material. The process comprises contacting the polymeric material with a metal salt solution. In a preferred embodiment, the polymeric material is porous and comprises at least part of the colloidal metal particles within the pores therein. More preferably, the polymeric material is in microparticle form and is suitable for injection or implantation into a human. In another preferred embodiment, the process comprises a step of heating a metal salt solution containing polymeric material at a temperature and for a time sufficient to associate the metal particles with the polymeric material. In another preferred embodiment, the process further comprises a step of mixing a reducing agent with the metal salt solution or irradiating the mixture with an irradiation source such as ultraviolet light. In a more preferred embodiment of the present invention's process, the metal salt solution is gold chloride (HAuCl₄) having a concentration ranging from about 0.1 μl to about 5 g/l.

In yet another aspect, the present invention is directed to a process of associating colloidal metal particles with a polymeric material that comprises contacting a polymeric material with a colloidal metal solution. Preferably, the polymeric material is porous and comprises at least pat of the colloidal metal particles within the pores therein. In another preferred embodiment, the polymeric material is in microparticle form having dimensions ranging from. In yet another preferred embodiment, the process comprises packing polymeric material, preferably, in porous microparticle form, in a column and perfusing the column with a colloidal metal solution. More preferably for this process, the colloidal metal particles have diameters that are smaller than the sizes of the pores, as measured by the cross section dimension.

The present invention further relates to a process of associating colloidal metal particles with a polymeric material by introducing colloidal metal particles into the initial polymerization solution or suspension of polymeric material. Preferably, the polymeric material is porous and comprises at least part of the colloidal metal particles within the pores therein.

According to this process, colloidal metals can be introduced either as colloidal metal solutions or as metal salt solutions. The process further enables colloidal metals particles that are larger than the pores of the polymeric material to be trapped within the pores, resulting in metal particles that are more tightly attached to the polymers. In a specific embodiment, the initial polymerization solution or suspension for the polymeric material comprises N-tris-hydroxy-methyl-methylacrylamide, diethylaninoethylacrylamide, and N,N-methylene-bis-acrylamide.

In another aspect, the present invention provides an injectable composition that comprises polymeric microparticles associated with colloidal metal particles and a biocompatible carrier. In a preferred embodiment, the injectable composition comprises microparticles that are porous and having at least part of the colloidal metal particles deposited within the pores therein.

In another preferred embodiment of the injectable composition, the microparticles comprise one or more polymers selected from the group consisting of acrylics, vinyls, acetals, allyls, cellulosics, polyamides, polycarbonate, polyesters, polyimide, polyolefins, polyurethanes, silicones, styrenics, and polysaccharides. In yet another preferred embodiment, the microparticles comprise an elastomer, a hydrogel, a water swellable polymer, or combinations thereof.

In another preferred embodiment, the injectable composition comprises microparticles that are substantially spheric micropheres suitable for one or more of dermal augmentation, tissue bulking, and embolization. More preferably, the microspheres comprise a hydrogel associated with colloidal gold particles and are suitable for injection or implantation into a human. In a most preferred embodiment, the microspheres have diameters ranging from about 10 μm to about 2000 μm, useful for embolization, and comprise a hydrophilic acrylic copolymer comprising, in copolymerized form, about 25 to about 98%, by weight, of a neutral hydrophilic acrylic monomer, about 2 to about 50%, by weight, of a difunctional monomer, and about 0 to about 50%, by weight, of one or more monomers having a cationic charge. Further, the microspheres may also comprise one or more cell adhesion promoters selected from the group consisting of collagen, gelatin, glucosaminoglycans, fibronectin, lectins, polycations, natural biological cell adhesion agents or synthetic biological cell adhesion agents.

In yet another aspect, the present invention provides a method of prophylactic, therapeutic, or cosmetic treatment of a mammal, which comprises administering to said mammal polymeric microparticles associated with colloidal metal particles. In a preferred embodiment, the administration is by means of injection through a syringe or a catheter. The method of treatment encompassed by the present invention includes one or more of dermal augmentation, tissue bulking, embolization, drug delivery, and treatment of gastroesophageal reflux disease, urinary incontinence, and vesicoureteral reflux disease.

The present invention further provides a kit for performing a prophylactic, therapeutic, or cosmetic treatment of a mammal. The kit comprises a sterile container and sterile and biocompatible polymeric microparticles associated with colloidal metal particles. In another embodiment, the present invention provides a kit for performing a prophylactic, therapeutic, or cosmetic treatment of a mammal that comprises a needle or a catheter, means for injecting a liquid based composition through said needle or catheter, and sterile and biocompatible polymeric microparticles associated with colloidal metal particles.

### DETAILED DESCRIPTION OF THE INVENTION

The present invention provides a unique and valuable system useful for labeling, controlling, and tracking implantable or injectable polymeric materials, especially microparticles, that are used in vivo, especially in humans, for prophylactic, therapeutic, and/or cosmetic purposes. Specifically, the invention allows physicians, e.g. surgeons and radiologists, to safely and effectively controlling and tracking the labeled materials during and after administration into the body. Therefore, the invention provides polymeric materials, especially microparticles, that are associated with colloidal metal particles, especially colloidal gold particles, which are visible under regular light through naked eye and optionally detectable by radio imaging and/or magnetic resonance imaging instruments. The invention also provides methods and processes of associating the polymeric materials, especially porous polymeric materials, with colloidal metal particles. The invention further provides injectable solutions and kits that comprise polymeric microparticles associated with colloidal metal particles. Moreover, the invention provides methods of prophylactic, therapeutic and cosmetic treatment of various conditions in a mammal by administering to the mammal microparticles associated with colloidal metals.

As used in the present invention, the term “implant” means a substance that is placed or embedded at least in part within the tissue of a mammal. When a substance is “implantable,” it is capable of being placed or embedded within the tissue through whatever means. For example, within the meaning of the present invention, a piece of traditional prosthetic device is an implant. So are substances, such as microparticles, that are placed within the dermal tissue of a mammal.

As used in the present invention, the term “embolization” means the occlusion or blockage of a blood vessel. The occlusion or blockage may occur either due to blood clots or emboli as a result of a physiological condition or due to an artificial act of embolic materials. In this regard, according to the present invention, an embolus is different from an implant.

As used in the present invention, the term “injectable” means capable of being administered, delivered or carried into the body via a needle, a catheter, or other similar ways.

As used in the present invention, “microparticles” means polymer or combinations of polymers made into bodies of various sizes. The microparticles can be in any shape, although they are often in substantially spherical shape, in which case the microparticles are referred to as “microspheres” or “microbeads.”

“Substantially spherical,” as used in the present invention generally means a shape that is close to a perfect sphere, which is defined as a volume that presents the lowest external surface area. Specifically, “substantially spherical” in the present invention means, when viewing any cross-section of the particle, the difference between the average major diameter and the average minor diameter is less than 20%, preferably less than 10%. The surfaces of the microspheres of the present invention preferably appear smooth under magnification of up to 1000 times. The microspheres of the present invention may comprise, in addition to the particles, other materials as described and defined herein.

“Dermal augmentation,” as used in the present invention refers to any change of the natural state of a mammal's skin and related areas due to external acts. The areas that may be changed by dermal augmentation include, but not limited to, epidermis, dermis, subcutaneous layer, fat, arrector pill muscle, hair shaft, sweat pore, and sebaceous gland.

“Tissue bulking,” as used in the present invention refers to any change of the natural state of a mammal's non-dermal soft tissues due to external acts or effects. The tissues encompassed by the invention include, but not limited to, muscle tissues, connective tissues, fats, and, nerve tissues. The tissues encompassed by the present invention may be part of many organs or body parts including, but not limited to, the sphincter, the bladder sphincter and urethra.

As used in the present invention, “associated with” means the condition in which two or more substances having any type of physical contact. For example, when a polymeric material is “associated with” colloidal metal particles, the metal particles may be deposited on the surface of the polymeric material, within the material, or, if the material is porous, within the pores of the material, through any type of physical or chemical interactions such as through covalent bond, ionic bond, or van der Waal's bond, or through impregnating, intercalating, or absorbing. According to the present invention, when a polymeric material is associated with colloidal metal particles, it is “labeled” with the colloidal metal particles.

Polymeric Materials Comprising Colloidal Metals Particles

In one aspect, the present invention is directed to a polymeric material that comprises colloidal metal particles. The polymeric material of the present invention includes synthetic and natural polymers. Preferably, the polymeric material is porous synthetic polymeric material and comprises at least part of the colloidal metal particles within the pores therein. In a preferred embodiment of the present invention, the material comprises one or more polymers selected from the group consisting of acrylics, vinyls, acetals, allyls, cellulosics, polyamides, polycarbonate, polyesters, polyimide, polyolefins, polyurethanes, silicones, styrenics, and polysaccharides. In another preferred embodiment, the polymeric material of the present invention is or is made to be an elastomer, a hydrogel, a water swellable polymer, or combinations thereof.

According to the present invention, the metal containing polymeric materials may be used in any medical applications, but they are especially suitable as implantable and/or injectable devices including, but not limited to, prosthetic devices, injectable dermal augmentation or tissue bulking materials. In a more preferred embodiment of the present invention, the colloidal metal labeled polymeric material is in microparticle form and useful as emboli for prophylactic or therapeutic embolizations. Therefore, the polymeric materials of the present invention are particularly suitable in injectable implantations or embolizations as small particles, such as microparticles, microbeads or microspheres. These microparticles are usually difficult to control or manipulate before or during injection because they are usually small in the amount used and, in many cases, transparent under regular light. The microparticles are also difficult control and monitor after the injection because of their sizes. The present invention makes it possible for the microparticles to be readily visible during the injection by associating colloidal metal particles with the material so that the material are visible under regular light and optionally detectable through radiological techniques.

Many types of polymeric microparticles or microspheres, either for tissue bulking, dermal augmentation, or embolization purposes, are suitable for the present invention. For example, the microparticles disclosed in U.S. Pat. Nos. 4,657,553; 4,999,188; 5,007,940; 5,092,883; 5,344,452 5,571,182; 5,635,215; 5,648,100; 5,785,997; 5,798,096; and 5,995,108 are encompassed by the present invention as polymeric materials that can be associated with colloidal metal particles according to the present invention. The above U.S. patents are herein specifically incorporated by reference. Also incorporated by references are U.S. patent application Ser. Nos. 09/263,773; 09/419,114; 09/528,990; 09/528,989; and 09/528,991, PCT applications PCT/US01/09618; PCT/US01/08258; PCT/US01/09619, and Japanese laid open patent application 6-56676, wherein microparticles, compositions, and/or uses thereof are disclosed.

In a preferred embodiment of the present invention, the polymeric material comprises an acrylic polymer. Because of its wide applications in medical implantable devices, polymethacrylates such as poly(methyl methacrylate) and poly(2-hydroxyethyl methacrylate) are especially suitable for the present invention.

In another preferred embodiment of the present invention, the porous polymeric materials comprise microbeads or microparticles based on a biocompatible, hydrophilic, substantially spherical, and non-toxic polymers. The microspheres are injectable ana/or implantable and not capable of being digested or eliminated through the mammal's immune or lymphatic system. More preferably, the hydrophilic copolymers usable for this application are those of the acrylic family such as polyacrylamides and their derivatives, polyacrylates and their derivatives as well as polyallyl and polyvinyl compounds. All of these polymers are preferably crosslinked so as to be stable and non-resorbable.

In a particularly preferred embodiment of the present invention, the microparticle comprises a polymeric material that comprises a hydrophilic acrylic copolymer, which contains, in copolymerized form, about 25 to about 98%, by weight, of a neutral hydrophilic acrylic monomer, about 2 to about 50%, by weight, of a difunctional monomer, and about 0 to about 50% by weight of one or more monomers having a cationic charge. More preferably, the neutral hydrophilic acrylic monomer is selected from the group consisting of acrylamides, methactylamides and hydroxymethylmethacrylate; the difunctional monomer is selected from the group consisting of N,N′-methylene-bis-acrylamide, N′,N′-diallyltartradiamide, and glyoxal-bis-acrylamide; and the monomer having a cationic charge is a monomer that has a tertiary and/or quaternary amine function.

In addition, the microparticle may optionally comprise one or more cell adhesion promoters selected from the group consisting of collagen, gelatin, glucosaminoglycans, fibronectin, lectins, polycations, natural biological cell adhesion agents or synthetic biological cell adhesion agents.

In another particularly preferred embodiment of the present invention, the polymeric material comprises poly (vinyl alcohol). Polyvinylalcohol particles are the most common material used to date in a variety of embolization applications. However, their usually irregular in shape and, thus, have numerous drawbacks, and can in certain circumstances even led to deaths. WO 00/23054, the content of which is incorporated by reference, discloses substantially spherical shaped microspheres comprising cross-linked PVA. The microspheres described therein has certain advantages in embolization. For example, due to their spherical shape or substantially spherical shape, microspheres can properly and completely occlude artery lumen because they can establish complete contact with all the surface of the artery which is cylindrical. In addition, the microspheres can be easily calibrated, and samples or suspensions containing these microspheres will not block or clog catheters because they always have the same dimension regardless of their space orientation in the catheter. The invention described herein encompasses PVA microspheres useful for tissue bulking and/or embolization. The PVA microspheres preferably comprise crosslinked polyvinylalcohol.

Preferred diameters for the microspheres depend on the type of tissue bulking or embolization to be performed and can be readily determined by the skilled artisans. The microspheres of the present invention can be in the form of dry powder or hydrogel. In a preferred embodiment, the present invention encompasses microspheres, which comprise in crosslinked and hydrogel form, from about 0.5% to about 20% cross-linked poly(vinyl alcohol) by weight. In other embodiments, the crosslinked polyvinylalcohol microspheres may further comprise one or more of a cell adhesion promoter or a marking agent other than the colloidal metal.

The polymeric material of the present invention, when in microparticle form, preferably have dimensions ranging from about 1 μm to about 2000 μm. Preferably, the microparticles are substantially spherical microspheres with diameters ranging from about 10 μm to about 2000 μm, more preferably, from about 40 μm to about 1200 μm.

According to a preferred embodiment of the present invention, the polymeric material contains or is made to contain pores. Preferably, the material comprises pores both on the surface and within. The pores contained within the polymeric material of the present invention have sizes, measured in cross-section diameters, ranging from about 1 nm to about 10 μm and, preferably, from about 1 mm to about 1000 nm. The lengths of the pores vary depending on the dimensions of the material. The pores facilitate the impregnation of and actually contain the colloidal metal particles, which are preferably trapped within the pores.

The porous polymeric material of the present invention preferably contains within the pores colloidal metal particles that have dimensions ranging from about 1 nm to about 1000 nm, more preferably, from about 1 nm to about 500 nm. The present invention contemplates mostly metals such as gold, anti platinum because they are non-toxic and biocompatible. However other metal are part of the invention whenever they can be transformed into metal colloidal particles as described above. The metal is preferably selected from the group consisting of gold, silver, platinum, copper, titanium and chromium. Among the metal particles, colloidal gold particles give the polymeric material of the present invention a distinctive red or red-like color, which makes the material readily visible under regular light, as well as by radiological imaging techniques. The impregnation of the metal particles within the polymers are the results of either direct deposition of colloidal metal particles on the porous polymeric material or a reduction process from a metal salt solution.

In a particularly preferred embodiment, the present invention provides a substantially spherical microparticle, or a microsphere, which comprises a hydrogel associated with colloidal gold particles, wherein the microsphere is suitable for injection or implantation into a human. In a more preferred embodiment, the present invention provides a microsphere having a diameter ranging between about 10 μm and about 2000 μm, useful for embolization, which comprises a hydrophilic acrylic copolymer associated with colloidal gold particles, wherein the hydrophilic acrylic copolymer comprises, in copolymerized form, about 25 to about 98%, by weight, of a neutral hydrophilic acrylic monomer, about 2 to about 50%, by weight, of a difunctional monomer, and about 0 to about 50%, by weight, of one or more monomers having a cationic charge.

The microsphere of the present invention may also comprise one or more cell adhesion promoters selected from the group consisting of collagen, gelatin, glucosaminoglycans, fibronectin, lectins, polycations, natural biological cell adhesion agents or synthetic biological cell adhesion agents. Further, the microsphere may optionally comprise a marking agent selected from the group consisting of dyes, imaging agents, and contrasting agents.

Processes of Associating Polymeric Materials with Colloidal Metal Particles

Another aspect of the present invention relates to processes of associating colloidal metal particles with the polymeric material. According to the present invention, the association process can be accomplished in at least three ways. First, colloidal metal particles can be associated with the polymeric materials through the reduction of a metal salt. Second, the metal particles can be deposited on and/or within the polymeric material through direct contact between the material and a colloidal metal solution. Third, the metal containing polymeric material can be produced by introducing a metal salt or colloidal metal solution into the initial polymerization solution or suspension of the polymeric material. In all three methods, the colloidal metal particles are preferably permanently associated on the polymeric materials, enable better detection and control of such materials in implantation applications. The various polymeric materials mentioned above are suitable for the association processes of the present invention.

According to the present invention, colloidal metal particles can be associated with a polymeric material by contacting the polymeric material with a metal salt solution for a time and at a temperature sufficient to reduce the metal salt into metal particles that are deposited on or within the polymeric material. In a preferred embodiment of the present invention, the polymeric material is porous and that the process enables the porous materials to comprise at least part of the colloidal metal particles within the pores of the material. In such cases, the sizes of the metal particles may either be larger or smaller than the sizes of the pores of the material, as measured by the cross-sections of the pores.

The associating process, according to the present invention, can be accelerated by heating the metal salt solution, preferably to boiling temperature. The process can be further accelerated by the addition of a reducing agent. Any agent that is known to have the ability to reduce a metal salt into metal particles can be used for this purpose. Preferred reducing agents include sodium citrate, ascorbic acid, phosphorous derivatives, tannic acid, citric acid, and combinations thereof. Another way of accelerating the reduction process is irradiation of the mixture the polymeric material and the metal salt solution. Preferred source of irradiation includes ultraviolet light such as that from a mercury lamp. After the impregnation/deposition processes, the polymeric material is preferably washed and/or filtered with water or saline to remove any non-deposited materials.

In a preferred embodiment of the present invention's process, the metal salt solution is gold chloride (HAuCl₄) having a concentration ranging from about 0.1 μl to about 5 g/l. More preferably, the process comprises heating the gold chloride solution containing the polymeric material, preferably to boiling temperature. Further, the addition of a reducing agent could accelerate the impregnation process, so could irradiation from source such as ultraviolet light, as discussed above.

The present invention also provides a process of associating colloidal metal particles with a polymeric material by contacting the polymeric material with a colloidal metal solution. In a preferred embodiment of the present invention, the polymeric material is porous and that the process enables the porous materials to comprise at least part of the colloidal metal particles within the pores of the material. In such a process, the sizes of the colloidal metal particles are preferably smaller than the sizes of the pores, as measured by the dimension of the cross sections of the pores.

In another preferred embodiment, the polymeric material is in microparticle form and has dimensions ranging from about 1 μm to about 2000 μm. A more preferred process for this direct deposition of colloidal metal particles comprises packing the polymeric material, such as microparticles, in a column and perfusing the column with the colloidal metal solution. This process can be preferably followed by rinsing the column with water or saline. When colloidal metal particles are used for porous materials, the particles are preferably of sizes smaller than the pores of the polymeric material. They also should be preferably suspended with a surfactant to maintain in a dissociated form.

According to the present invention, a third way of associating colloidal metal particles with the polymeric material comprises adding colloidal metal particles or a metal salt solution into the initial polymerization solution or suspension for the polymeric material. In a preferred embodiment of the present invention, the resultant polymeric material is porous and that the process enables the porous materials to comprise at least part of the colloidal metal particles within the pores of the material.

In such a polymerization/association process, there is preferably no change in the polymerization process for the polymeric material itself. Therefore, any polymerization process that produces a polymeric material can be incorporated into the process of the present invention by adding a solution of metal salt or colloidal metal into the initial polymerization solution or suspension. For example, polymerization processes disclosed references incorporated herein are encompassed by the present invention. In particular, polymerization processes disclosed in U.S. Pat. No. 5,635,215 for producing acrylic microspheres and in WO 00/23054 for producing PVA microspheres can be incorporated into the process of the present invention to produce hydrophilic acrylic microspheres or PVA microspheres containing colloidal metal particles. When the initial polymerization solution or suspension is transformed into a acrylic or PVA microsphere, preferably in hydrogel form, the colloidal particles are trapped within the polymer network and cannot be released any longer. In this case they are located inside the polymer pores and confer a colored aspect to the beads as a function of the nature of the metal. In case of a porous polymeric material, the resulting metal containing material from this process may contain colloidal metal particles that are larger in size than the sizes of the pores, as measured by the dimensions of the cross sections of the pores.

Injectable Compositions, Kits, and Methods of Use

The present invention further encompasses injectable compositions, kits, and methods of use in connection with the colloidal metal containing polymeric materials disclosed above.

In one embodiment, there is provided an injectable composition that comprises polymeric microparticles associated with colloidal metal particles and a biocompatible carrier. The various embodiments of the colloidal metal containing microparticles disclosed herein are suitable for the injectable compositions. In addition, the microparticles and biocompatible carriers disclosed in the various U.S. patents, U.S. and PCT patent applications incorporated by references herein are also suitable for the injectable compositions of the present invention.

In another embodiment, the present invention provides a method of prophylactic, therapeutic, or cosmetic treatment of a mammal, preferably a human, which comprises administering to the mammal polymeric microparticles associated with colloidal metal particles. Due to the unique characters of the microparticles of the present invention, the administration is capable of being well controlled and/or manipulated both before and after the process, as the microparticles are readily visible under regular light before the administration and optionally using radio imaging and/or magnetic resonance techniques after the administration.

Suitable treatment encompassed by the present invention includes dermal augmentation, tissue bulking, embolization, drug delivery, and treatment of gastroesophageal reflux disease, urinary incontinence, and vesicoureteral reflux disease. The administration according to the method of treatment of the present invention is preferably carried out by means of injection through a syringe or a catheter. In this regard, the methods of treatment disclosed in the U.S. patents, U.S. and PCT patent applications incorporated by reference herein are also encompassed by the present invention's method.

Finally, the present invention provides a kit for performing a prophylactic, therapeutic, or cosmetic treatment of a mammal. The kit preferably comprises a sterile container and sterile and biocompatible polymeric microparticles associated with colloidal metal particles. In another preferred embodiment, the kit of the present invention for performing a prophylactic, therapeutic, or cosmetic treatment of a mammal comprises a needle or a catheter; means for injecting a liquid based composition through said needle or catheter; and sterile and biocompatible polymeric microparticles associated with colloidal metal particles. In this regard, the various embodiments of the microparticles disclosed herein and the various embodiments disclosed in the U.S. patents, U.S. and PCT patent applications incorporated by reference herein are also encompassed by the present invention's kit.

The present is further defined by reference to the following examples that describe in detail the preparation of colloidal metal labeled microparticles. In addition, the examples disclosed in the U.S. patents and U.S. and PCT patent applications incorporated by reference herein are also illustrative of the present invention. The examples should in no way limit the scope of the present invention. It will be apparent to those skilled in the art that many modifications, both to materials and methods, may be practiced without departing from the purpose and scope of this invention.

### EXAMPLES

### Example 1

### Gold Staining of Embolic Spherical Material Constituted of a Synthetic Polymer Containing Crosslinked Collagen (e.g. Embosphere®)

Solutions of HAuCl_{4 (}0.1 to 5.0 g/l) (Solution I) and of sodium citrate as reducing agent (1% by weight) (Solution II) were prepared. A suspension of Embosphere® (10 ml) and Solution I (20 ml of the desired concentration) were heated to boiling and then 2 ml of Solution II was added. After 10 minutes the solution and Embosphere® turned to red, indicating the formation of gold colloidal particles within the solid material network. The beads were then filtered and washed several times with water and saline. Similar results were obtained when using other reducing agents, instead of sodium citrate, such as ascorbic acid, phosphorous derivatives or sodium citrate and tannic acid.

### Example 2

### Gold Staining of PVA Particles (Spherical or Irregular) as Embolic Material

Solutions of 3 g/l of HAuCl₄ (Solution I) and of 1% ascorbic acid as reducing agent (Solution H) were prepared. 10 ml of a suspension of PVA solid particles was mixed with 20 ml of solution and heated to boiling. To the boiling suspension, 2 ml of Solution II was added. After 10 minutes, the suspension of embolic material turned to red, indicating the formation of gold colloidal particles within the solid material network. The beads were then filtered and washed extensively with water and saline. Similar results were obtained using other reducing agents, instead of ascorbic acid, such as citric acid, tannic acid, and phosphorous derivatives.

### Example 3

### Embolic Solid Material Staining Without Reducing Agents

The same procedure was used as described in Example 1, but without a reducing agent. The suspension of Embosphere® or PVA particles with Solution I were heated to boiling for an extensive period of time (15 minutes or more). The beads and the solution appeared red-brown, which confirmed the formation of gold particles within the solid material network. The beads were then treated with the same manner as described in Examples 1 and 2. The reduction of gold could also be accomplished by irradiation of the samples with a mercury lamp for about 48 hours at 25° C.

### Example 4

### Staining Procedure Concomitant to Bead Preparation by Acrylic Polymerization

In a beaker containing 100 ml of HAuCl₄ solution at a concentration of 3 g/liter, 29 g of sodium chloride and 13.5 g of sodium acetate were dissolved. 200 ml of glycerol was added and then the pH was adjusted between 5.9 and 6.1. Then 45 g of N-tris-hydroxy-methyl-methylacrylamide, 17.5 g of diethylaminoethylacrylamide and 5 g of N,N-methylene-bis-acrylamide were added. Once the solution was at 60° C., 60 ml of a water solution containing 10 g of gelatin was added. The total volume of the mixture was adjusted to 500 ml by addition of hot water. To this solution 10 ml of a 700 mg ammonium persulfate solution and 2 ml of N,N,N′,N′-tetramethylenediamine were added. The resulting mixture was rapidly stirred to mix all ingredients together and poured into double volume of stirred paraffin oil at 58° C. After a few minutes, the polymerization reaction of acrylic monomers was manifested by an increase of temperature. To the emulsion 400 ml of sodium citrate solution (1% by weight) was then added and the suspension heated to 70-80° C. Resulting red beads were recovered by decanting, washed carefully, sieved and sterilized in an autoclave in a physiological saline medium.

### Example 5

### Staining Procedure Concomitant to Bead Preparation by Crosslinking

To an aqueous solution of PVA (50 g in 300 ml), glutaraldehyde (10 ml of 25% aqueous solution) and HAuCl₄ solution (100 ml of 3 g/l) were added under stirring at 55° C. This solution was then dispersed in a medium consisting of 1000 ml of paraffin oil and 1 ml of Arlacel®. Thionyl chloride (10 ml) was then introduced to the emulsion and kept at 25° C. under stirring (180 rpm) for five hours. To the suspension, 400 ml of a solution of sodium citrate (1% by weight) was then added and the mixture heated at 70-90 C for one hour. Resulted crosslinked PVA microsphere were recovered by decanting. They were washed, sieved and sterilized in an autoclave in a saline medium.

### Example 6

### Staining of Beaded Embolic Agent with Colloidal Platinum

A solutions of H₂PtCl₆ at a concentration of 5.3 g/l was prepared in water under stirring (Solution I). A second solution of saturated hydrazine sulfate in water was also prepared (Solution II). To a suspension of 10 ml of embolic beads (e.g., Embosphere®) 20 ml of Solution I was added under stirring. The resulting suspension was then heated to boiling temperature and then added with 5 ml of Solution II. After 10 minutes agitation, the embolic materials turned to grey, indicating the formation of colloidal platinum nanoparticles. The beads were then filtered and washed several times with water and physiological saline.

### Example 7

### Staining of a Commercially Available Embolic Material

The same procedure was used as described in Example 1, but Ivalon® was used instead of Embosphere®. The suspension of Ivalon® irregular particles with Solution 1 (HAuCl₄, 3 g/l) was heated to boiling temperature and then 2 ml of Solution 11 (1% sodium citrate in water) was added. After 10 minutes of agitation, the suspension turned to red-brown, indicating the formation of gold colloidal particles in the Ivalon® particles. The particles were then filtered and washed several times with water and saline. Similar results were obtained when using other reducing agents instead of sodium citrate such as ascorbic acid, phosphorous derivatives or sodium citrate/tannic acid. The reduction to colloidal gold could also be made by irradiation of the suspension with a mercury lamp for about 48 hours at 25° C.

### Example 8

### Staining of Embolic Biodegradable Embolic Particles

This process applies to embolic microparticles (irregular arid spherical) composed of polysaccharide and/or proteins (e.g., albumin). The same procedure was used as described in Example 1, but biodegradable solid embolic is used instead of Embosphere®. 10 ml particles were put in suspension with 20 ml of an aqueous Solution I of HAuCl₆ at 3 g/l. The mixture was then heated to boiling temperature and then 2 ml of 1% sodium citrate solution in water was added. After 10 minutes agitation, the suspension turned to red-brown indicating the formation of gold colloidal particles inside the embolic material. The particles were then filtered and washed several times with water and saline. Similar results were obtained using other reducing agents, instead of sodium citrate, such as ascorbic acid, phosphorous derivatives or sodium citrate/tannic acid. The reduction to colloidal gold could also be made by irradiation of the suspension with a mercury lamp for up to about 48 hours at 25° C.

### Example 9

### Staining of Solid Embolic Material with Gold Colloidal Particles

This method of staining applies to embolic material that has porous structure with pores larger than 10 nm in diameter. Embolic material in aqueous suspension was packed in a glass column. Through the column a colloidal solution of gold was perfused. Colloidal particles that had a size smaller than the pores of the solid embolic material were trapped within the embolic pore network. The excess of gold colloidal particles or colloidal particles that were larger man the pores of the solid embolic were washed out the column by means of a physiological buffer. After the treatment the embolic material showed a red like color, indicating the presence of colloidal gold entrapped within the pore network.

### Example 10

### Injectable Compositions Containing Gold Labeled Embosphere®

Gold labeled Embosphere® microspheres, as described in Examples 1, 3 and 4 are washed with normal saline and then sterilized by autoclave. The resultant microspheres are mixed with non-pyrogenic, sterile, physiological saline in ratios ranging from about 0.05 ml microspheres/ml saline to about 0.5 ml microspheres/ml saline.

### Example 11

### Kits Containing Gold Labeled Embosphere®

A total amount of 8 ml of sterile injectable composition as described in Example 10 is transferred, under sterile condition, into a glass vial of 10 ml in capacity and having a stopper sealed by an aluminum cap equipped with a colored tag.

The embodiments of the present invention described above are intended to be merely exemplary and those skilled in the art will recognize, or be able to ascertain using no more than routine experimentation, numerous equivalents to the specific procedures described herein. All such equivalents are considered to be within the scope of the present invention and are covered by the following claims.

The contents of all references described herein are hereby incorporated by reference. Other embodiments are within the following claims.

1-59. (canceled)

60. An implant comprising a polymeric material associated with colloidal metal particles.

61. The implant of claim 60, wherein the polymeric material comprises one or more polymers selected from the group consisting of acrylics, vinyls, acetals, allyls, cellulosics, polyamides, polycarbonate, polyesters, polyimide, polyolefins, polyurethanes, silicones, styrenics, and polysaccharides.

62. The implant of claim 61, wherein the polymeric material is porous.

63. The implant of claim 62, wherein the polymeric material comprises at least part of the colloidal metal particles within the pores therein.

64. The implant of claim 63, wherein the polymeric material is suitable for implantation into a human.

65. A microparticle which comprises a polymeric material associated with colloidal metal particles, wherein the microparticle is suitable for injection or implantation into a human.

66. The microparticle of claim 65, wherein the polymeric material comprises one or more polymers selected from the group consisting of acrylics, vinyls, acetals, allyls, cellulosics, polyamides, polycarbonate, polyesters, polyimide, polyolefins, polyurethanes, silicones, styrenics, and polysaccharides.

67. The microparticle of claim 66, wherein the material is porous.

68. The microparticle of claim 67, wherein the material comprises at least part of the colloidal metal particles within the pores therein.

69. The microparticle of claim 66, wherein the polymeric material is an elastomer, a hydrogel, a water swellable polymer, or combinations thereof.

70. The microparticle of claim 66, wherein the polymeric material comprises a hydrophilic acrylic copolymer.

71. The microparticle of claim 70, wherein the hydrophilic acrylic copolymer comprises, in copolymerized form, about 25 to about 98%, by weight, of a neutral hydrophilic acrylic monomer, about 2 to about 50%, by weight, of a difunctional monomer, and about 0 to about 50%, by weight, of one or more monomers having a cationic charge.

72. The microparticle of claim 71, wherein the neutral hydrophilic acrylic monomer is selected from the group consisting of acrylamides, methacrylamides and hydroxymethylmethacrylate.

73. The microparticle of claim 71, wherein the difunctional monomer is selected from the group consisting of N,N′-methylene-bis-acrylamide, N′,N′-diallyltartradiamide, and glyoxal-bis-acrylamide.

74. The microparticle of claim 71, wherein the monomer having a cationic charge is a monomer having a tertiary and/or quaternary amine function.

75. The microparticle of claim 71, wherein the microparticle further comprises one or more cell adhesion promoters selected from the group consisting of collagen, gelatin, glucosaminoglycans, fibronectin, lectins, polycations, natural biological cell adhesion agents or synthetic biological cell adhesion agents.

76. The microparticle of claim 75, wherein the microparticle further comprises a marking agent selected from the group consisting of dyes, imaging agents, and contrasting agents.

77. The microparticle of claim 66, wherein the polymeric material is a polymethacrylate.

78. The microparticle of claim 77, wherein the polymeric material is poly(methyl methacrylate) or poly(2-hydroxyethyl methacrylate).

79. The microparticle of claim 66, wherein the polymeric material is cross-linked poly(vinyl alcohol).

80. The microparticle of claim 66, wherein the microparticle has dimensions ranging from about 1 μm to about 2000 μm.

81. The microparticle of claim 80, wherein the microparticle is a substantially spherical microsphere have a diameter ranging from about 10 μm to about 2000 μm.

82. The microparticle of claim 66, wherein the microparticle is suitable for tissue bulking or dermal augmentation purposes.

83. The microparticle of claim 66, wherein the microparticle is suitable for therapeutic vascular embolization.

84. The microparticle of claim 67, wherein the polymeric material comprises pores both on the surface and within.

85. The microparticle of claim 84, wherein the pores have sizes ranging from about 1 nm to about 10 μm.

86. The microparticle of claim 65, wherein the metal is selected from the group consisting of gold, silver, platinum, copper, titanium and chromium.

87. The microparticle of claim 86, wherein the colloidal metal particles have dimensions ranging from about 1 nm to about 1000 nm.

88. The microparticle of claim 87, wherein the colloidal metal particles have dimensions ranging from about 1 nm to about 500 nm.

89. A microsphere which comprises a hydrogel associated with colloidal gold particles, wherein the microsphere is suitable for injection or implantation into a human.

90. A microsphere having a diameter ranging between about 10 μm and about 2000 μm, useful for embolization, which comprises a hydrophilic acrylic copolymer associated with colloidal gold particles, wherein the hydrophilic acrylic copolymer comprises, in copolymerized form, about 25 to about 98%, by weight, of a neutral hydrophilic acrylic monomer, about 2 to about 50%, by weight, of a difunctional monomer, and about 0 to about 50%, by weight, of one or more monomers having a cationic charge.

91. The microsphere of claim 90, wherein the microsphere further comprises one or more cell adhesion promoters selected from the group consisting of collagen, gelatin, glucosaminoglycans, fibronectin, lectins, polycations, natural biological cell adhesion agents or synthetic biological cell adhesion agents.

92. The microsphere of claim 90, wherein the microsphere further comprises a marking agent selected from the group consisting of dyes, imaging agents, and contrasting agents.

93. An injectable composition suitable for administration to a human comprising polymeric microparticles associated with colloidal metal particles and a biocompatible carrier.

94. The injectable composition of claim 93, wherein the microparticles comprise one or more polymers selected from the group consisting of acrylics, vinyls, acetals, allyls, cellulosics, polyamides, polycarbonate, polyesters, polyimide, polyolefins, polyurethanes, silicones, styrenics, and polysaccharides.

95. The injectable composition of claim 94, wherein the microparticles are substantially spherical microspheres suitable for one or more of dermal augmentation, tissue bulking, and embolization.

96. The injectable composition of claim 95, wherein the microspheres comprise a hydrogel associated with colloidal gold particles, wherein the microsphere is suitable for injection or implantation into a human.

97. The injectable composition of claim 96, wherein the microspheres have diameters ranging between about 10 μm and about 2000 μm, useful for embolization, and comprise a hydrophilic acrylic copolymer comprising, in copolymerized form, about 25 to about 98%, by weight, of a neutral hydrophilic acrylic monomer, about 2 to about 50%, by weight, of a difunctional monomer, and about 0 to about 50%, by weight, of one or more monomers having a cationic charge.

98. The injectable composition of claim 97, wherein the microspheres further comprise one or more cell adhesion promoters selected from the group consisting of collagen, gelatin, glucosaminoglycans, fibronectin, lectins, polycations, natural biological cell adhesion agents or synthetic biological cell adhesion agents.
